(19) United States
(12) Patent Application Publication
(10) Pub. No.: US 2026/0033856 A1
(43) Pub. Date: Feb. 5, 2026

(54) SERRATED ULTRASONIC CUTTING BLADE WITH VARIED TOOTH PITCH

(71) Applicant: Misonix, LLC, Farmingdale, NY (US)

(72) Inventors: Elad I. LEVY, Amherst, NY (US); Timothy O'CONNOR, Boca Raton, FL (US); Dan VOIC, Cedar Grove, NJ (US)

(21) Appl. No.: 19/296,789

(22) Filed: Aug. 11, 2025

Related U.S. Application Data
(63) Continuation of application No. 17/402,295, filed on Aug. 13, 2021, now Pat. No. 12,402,905.

(51) Int. Cl. A61B 17/32

(52) U.S. Cl. CPC A61B 17/320068; A61B 2017/320077

(57) ABSTRACT

An ultrasonic blade is provided along a convexly arcuate distal edge and at least one longitudinal edge with a continuous array of teeth including a first subset of teeth along the convexly arcuate distal edge and a second subset of teeth along the at least one straight longitudinal edge. The teeth along the convexly arcuate distal edge differ in tooth length and optionally in inter-tooth separation or pitch from the teeth along one or both of the longitudinal edges.

### CROSS-REFERENCE TO RELATED APPLICATIONS

This application is a continuation of U.S. patent application Ser. No. 17/402,295, filed Aug. 13, 2021, which is incorporated herein by reference in its entirety.

### FIELD OF THE INVENTION

This invention relates to an ultrasonic tool. More particularly, this invention relates to an ultrasonic cutting blade. The blade is particularly useful in a surgical application to cut tissue such as cartilage and bone.

### BACKGROUND OF THE INVENTION

In the field of orthopedics, the cutting of living bone is a prerequisite for many procedures. Such procedures include the reconstruction of damaged tissue structures due to accidents, the grafting of healthy bone into areas damaged by disease, or the correction of congenital facial abnormalities like a receding chin line. Over several centuries, these tasks were performed through the utilization of devices called bone saws.

Traditional bone saws are categorized into several basic categories. Hand powered saws or drills are just that, hand held devices which require the operator to move the device in a fashion similar to that used for carpentry tools. Powered devices, whether electric or pneumatic, are of either the reciprocating or rotary type. The reciprocating devices use a flat, sword like blade where the back and forth motion is provided by a motor instead of the hand. The rotary devices use a rotating motor to spin a drill bit or a blade that has teeth arranged around its circumference similar to a table saw blade. All of these traditional bone saws are used today in medical procedures around the world.

While traditional saws are functional, they have many disadvantages. With either the band or reciprocating saws, for instance, it is not easy to initiate and direct a cut. A cut must start from an edge or, alternatively, a starting hole must be used. To create a starting hole, a drill or similar instrument is operated to bore into the bone. Subsequently, a cutting blade is inserted into the bored hole. The user can then proceed to cut. Alternatively, a rotary type blade may be used. However, when a rotary blade is used, the cut must follow a relatively straight path to prevent the blade from binding in the cut. With all blades the ability to create a curved or compound angle cut is extremely limited by the blade chosen. The relatively thick blades have a wide kerf, so that a significant thickness of the viable bone is lost in the cutting procedure. Physicians would like this width to be as thin as possible in most procedures where reconstruction is necessary.

Ultrasonic bone cutting blades as disclosed, for example, in U.S. Pat. Nos. 5,261,922; 6,379,371; 6,443,969; 6,763,673; 8,888,783; 9,387,005; 9,320,528; D680,218; and D667,117 overcome many of the disadvantages and problems discussed above. However room for improvement remains. One problem is that surgeons sometimes have difficulty in penetrating into particularly large and strong bone structures, including spinal vertebrae and spinal lamina and processes.

### OBJECTS OF THE INVENTION

It is an object of the present invention to provide an improved ultrasonic surgical tool or probe for ablating or dissecting osseous tissue and cartilage.

Another object of the present invention is to provide such an ultrasonic surgical tool or probe that more readily facilitates bone cutting procedures, particularly, but not exclusively, spinal bone cutting procedures.

Yet another object of the present invention is to provide such an ultrasonic surgical tool or probe that is less likely to cut non-osseous tissue than bone.

Yet another object of the present invention is to provide a surgical disc space preparation procedure that may be used to access spinal discs in surgical procedures.

These and other objects of the invention will be apparent from the drawings and descriptions herein. Although every object of the invention is attained in at least one embodiment of the invention, there is not necessarily any embodiment which attains all of the objects of the invention.

### SUMMARY OF THE INVENTION

An ultrasonic surgical tool in accordance with the present invention comprises a substantially planar blade body having a pair of opposed lateral surfaces, a pair of straight longitudinal edges, and a convexly arcuate distal edge contiguous with the straight longitudinal edges. A shank is integral on a distal side with the blade body and provided at a proximal side with a connector for operatively linking the blade to a source of ultrasonic mechanical vibrations. The blade body is provided along the convexly arcuate distal edge and at least one of the straight longitudinal edges with a continuous array of teeth including a first subset of teeth along the convexly arcuate distal edge and a second subset of teeth along the at least one straight longitudinal edge. The first subset of teeth exhibits a first tooth length and a first inter-tooth gap depth, while the second subset of teeth has a second tooth length and a second inter-tooth gap depth. The first tooth length differs from the second tooth length, and the first inter-tooth gap depth differs from the second inter-tooth gap depth.

Another feature of an ultrasonic surgical tool in accordance with the present invention is that the first tooth length and the first inter-tooth gap depth are each uniform among the first subset of teeth, while the second tooth length and the second inter-tooth gap depth are each uniform among the second subset of teeth.

Pursuant to another feature of the present invention, the first tooth length and the first inter-tooth gap depth are smaller than the second tooth length and the second inter-tooth gap depth, respectively. Specifically, the first tooth length is between about 0.60 and about 0.85 times the second tooth length, and the first inter-tooth gap depth is between about 0.60 and about 0.85 times the second inter-tooth gap depth. Preferably, the first tooth length is about 0.80 times the second tooth length, and the first inter-tooth gap depth is about 0.80 times the second inter-tooth gap depth.

As indicated above, the first tooth length and the first inter-tooth gap depth may be uniform among the first subset of teeth, while the second tooth length and the second inter-tooth gap depth are uniform among the second subset of teeth. However, this is not invariably the case. For instance, the first tooth length and the first inter-tooth gap depth may vary from a minimum at an extreme distal tip of the arcuate distal edge and increase gradually on each side towards the respective longitudinal edge of the blade.

In a preferred embodiment of the invention, the teeth of the continuous array of teeth are all isometrically triangular and bear a common angle between opposing edges. In that event, where the characteristic tooth length of the first subset of teeth is smaller than the characteristic tooth length of the first subset of teeth, the pitch of the teeth of the first subset, along the distal edge of the blade, is necessarily smaller than the pitch of the teeth of the second subset, along one or two longitudinal edges of the blade.

Pursuant to this preferred embodiment, an ultrasonic surgical tool in accordance with the present invention comprises a substantially planar blade body having a pair of opposed lateral surfaces, a pair of straight longitudinal edges and a convexly arcuate distal edge contiguous with the straight longitudinal edges, with a shank integral on a distal side with the blade body being provided at a proximal side with a connector for operatively linking the blade to a source of ultrasonic mechanical vibrations. The blade body is provided along the convexly arcuate distal edge and at least one of the straight longitudinal edges with a continuous array of teeth including a first subset of teeth along the convexly arcuate distal edge and a second subset of teeth along the at least one of the straight longitudinal edges. The first subset of teeth exhibit a first inter-tooth separation or pitch and the second subset of teeth has a second inter-tooth separation, wherein the first inter-tooth separation or pitch differs from the second inter-tooth separation or pitch.

In accordance with a further feature of the present invention, the first inter-tooth separation or pitch is uniform among the first subset of teeth, and the second inter-tooth separation or pitch is uniform among the second subset of teeth.

Pursuant to a more specific feature of the present invention, the first inter-tooth separation or pitch is smaller than the second inter-tooth separation or pitch. The teeth of the continuous array of teeth are preferably all isometrically triangular and may bear a common angle between opposing edges.

Preferably, the first inter-tooth separation or pitch is between about 0.60 and about 0.85 times the second inter-tooth separation or pitch.

An ultrasonic tool in accordance with the present invention facilitates the performance of spinal surgeries, particularly by reducing the likelihood of damage to non-target soft tissue. It is believed that the smaller tooth length along the distal tip of the blade is less aggressive than longer tooth lengths against tissue not intended for removal, such as soft tissue of the spine.

While a uniform tooth sharpness (per the angle between relatively inclined tooth edges) is preferred, largely for manufacturing reasons, it is contemplated that the tooth angle may be reduced along the longitudinal edges to reduce the pitch and thereby increase the fineness of cutting and produce a smoother kerf.

### BRIEF DESCRIPTION OF THE DRAWINGS

FIG. 1 is a top plan view of an ultrasonic surgical tool or probe, particularly an ultrasonic surgical blade, in accordance with the present invention, a bottom plan view being identical to the top plan view.

FIG. 2 is a rear, left side and top perspective view of the ultrasonic surgical tool or probe of FIG. 1.

FIG. 3 is a front elevational view of the ultrasonic surgical tool or probe of FIGS. 1 and 2.

FIG. 4 is a rear elevational view of the ultrasonic surgical tool or probe of FIGS. 1-3.

FIG. 5 is a right side elevational view of the ultrasonic surgical tool or probe of FIGS. 1-4, a left side elevation view being identical thereto.

FIG. 6 is longitudinal axial cross-sectional view of the ultrasonic surgical tool or probe of FIGS. 1-5.

FIG. 7 is partial top or bottom plan view thereof, on an enlarged scale, showing detail VI in FIG. 1.

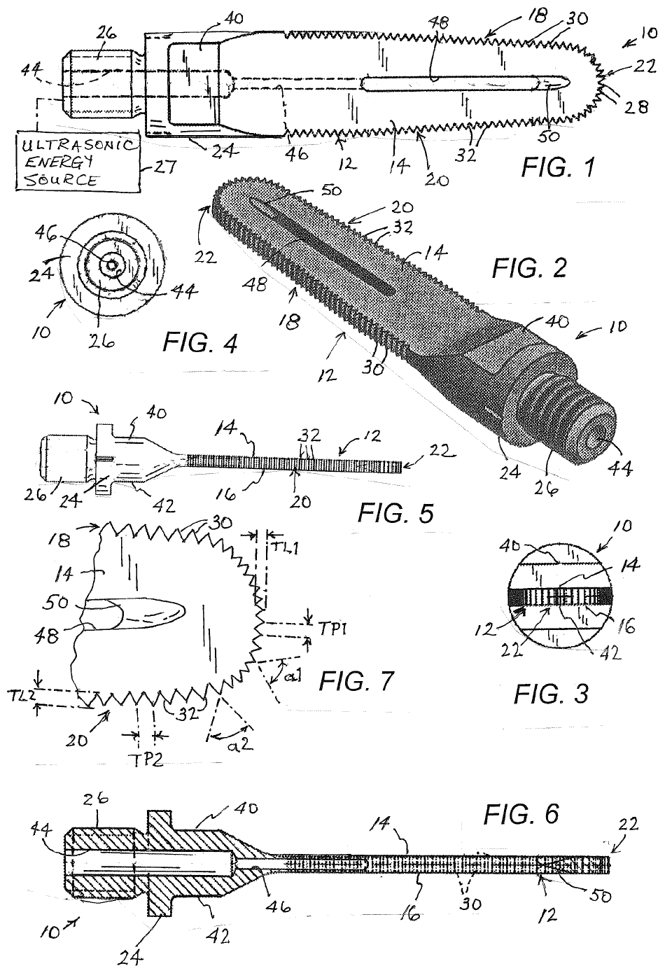

### DETAILED DESCRIPTION

As depicted in the drawings, an ultrasonic surgical tool or probe 10 includes a substantially planar blade body 12 having a pair of parallel opposed lateral surfaces or major faces 14 and 16, a pair of straight substantially longitudinal edges 18 and 20, and a convexly arcuate distal edge 22 contiguous and continuous with the straight longitudinal edges. Longitudinal edges 18 and 20 converge slightly towards one another at the distal end of blade body 12; longitudinal edges 18 and 20 are oriented at an angle of several degrees of arc relative to one another.

A transversely enlarged proximal portion or shank 24 is integral on a distal side with blade body 12 and provided on a proximal side with an externally threaded connector 26 for operatively coupling the blade body (“blade”) 12 to a source 27 of ultrasonic mechanical vibratory energy, particularly to a stack of piezoelectric crystal elements (not shown) and a waveform generator (not shown) that applies an ultrasonic-frequency voltage across the piezo-stack. Shank 24 is formed with a pair of opposed flats 40, 42 engageable by a wrench (not illustrated) for screwing the tool or probe 10 to an ultrasonic handpiece and particularly the piezoelectric crystal stack therein.

Shank 24 and blade body 12 are formed with co-linear channels, bores or lumens 44 and 46 of different diameters that communicate with one another and at a distal end with a through slot 48 extending longitudinally and axially along a portion of blade body 12. Liquid coolant, typically aqueous, is conveyed through channels 44 and 46 to slot 48 for maintaining the temperature of blade 12 and or adjacent biological tissue within a biologically safe range.

Blade body 12 is provided along convexly arcuate distal edge 22 and at least one but preferably both longitudinal edges 18 and 20 with a continuous array of teeth including a first subset of teeth 28 along convexly arcuate distal edge 22 and a second subset of teeth 30, 32 along longitudinal edges 18 and 20. Teeth 28 exhibit a first tooth length TL1 and an associated first inter-tooth gap depth (not designated) typically equal to one another, while teeth 30 and 32 have a second tooth length TL2 and a second inter-tooth gap depth (not designated) generally equal thereto. Tooth length TL1 differs from the tooth length TL2, and concomitantly the first inter-tooth gap depth differs from the second inter-tooth gap depth. A conically tapered surface 50 at a distal end of through slot 48 serves to distribute coolant to arcuate edge 22 along the length thereof.

Preferably, but not necessarily, tooth length TL1 and the associated inter-tooth gap depth are each uniform among teeth 28, while tooth length TL2 and the associated inter-tooth gap depth are each uniform among at least teeth 30 or 32. However, the lengths of teeth 28 and the associated first inter-tooth gap depth may vary from a minimum at an extreme distal tip of arcuate distal edge 22 and increase gradually on each side towards the respective longitudinal edge 30, 32 of blade body 12. Or one might provide teeth 30 and teeth 32 with different common tooth lengths TL2 and inter-tooth gap depths, where an application targets a region of different bone structures or densities. Preferably, tooth length TL1 and the inter-tooth gap depth of teeth 28 are respectively smaller than tooth length TL2 and the inter-tooth gap depth of teeth 30, 32. Specifically, tooth length TL1 lies between approximately 0.60 and approximately 0.85 times tooth length TL2, the associated inter-tooth gap depths exhibiting the same proportionality. Preferably, tooth length TL1 is about 0.80 times tooth length TL2, for instance, where tooth length TL1 is 0.0016 inch while tooth length TL2 is 0.0020 inch. Concomitantly, the inter-tooth gap depth of teeth 28 is 0.80 times the second inter-tooth gap depth of teeth 30 and/or 32.

Teeth 28, 30, 32 are preferably all isometrically triangular and bear a common angle a1, a2 between opposing edges. This geometric congruence simplifies manufacture. However, angles a1 and a2 may differ from one another. For instance, angle a1 may be larger than angle a2. Where the tooth length TL2 is unchanged, the reduction in the angle a2 and the consequent increase in sharpness of teeth 30, 32 correlates to a reduction in a pitch TP2 thereof. Such a reduction in pitch may be implemented to increase the fineness of cutting action of edges 30, 32.

Thus, an ultrasonic surgical tool in accordance with the present invention comprises planar blade body 12 with opposed lateral surfaces 14 and 16, straight longitudinal edges 18 and 20 and convexly arcuate distal edge 22 contiguous with edges 18 and 20, with shank 24 integral on a distal side with blade body 12 and provided with connector 26 for operatively linking blade 12 to vibration source 27. As described above, blade body 12 is provided along convexly arcuate distal edge 22 and at least one of longitudinal edges 18, 20 with a continuous array of teeth including teeth 28 along distal edge 22 and teeth 30, 32 along longitudinal edges 30, 32. Teeth 28 exhibit a first inter-tooth separation or pitch TP1 and teeth 30 and/or 32 has a second inter-tooth separation or pitch TP2 which may be different from inter-tooth separation or pitch TP1.

In a preferred embodiment, inter-tooth separation or pitch TP1 is uniform among teeth 28, while inter-tooth separation or pitch TP2 is uniform among teeth 30 and/or 32. Inter-tooth separation or pitch TP1 is smaller than inter-tooth separation or pitch TP2, where the teeth of the continuous array of teeth are all isometrically triangular and bear a common angle between opposing edges (a1=a2, exemplarily 60°). Alternatively, one might vary the manufacture, particularly the size of angles a1 and a2, so that pitches TP1 and TP2 are the same.

Ultrasonic tool or probe 10 enables a finer control of ablation in spinal surgery by reducing the action of the probe on tissues distal of blade body 12. The shorter tooth length TL1 renders arcuate edge 22 less aggressive, reducing the distal cutting action in relative to the rapidity of ultrasonic cutting along longitudinal edges 18 and 20.

Although the invention has been described in terms of particular embodiments and applications, one of ordinary skill in the art, in light of this teaching, can generate additional embodiments and modifications without departing from the spirit of or exceeding the scope of the claimed invention. For instance, the pitch and the tooth length (or depth or height) may gradually vary from characteristic values along edges 30 and 32 to different characteristic values along leading or distal edge 22. Alternatively, as indicated schematically in FIG. 7, the change may occur in a single step at a first given point between edges 18 and 22 and at another demarcated point between edges 20 and 22. Accordingly, it is to be understood that the drawings and descriptions herein are proffered by way of example to facilitate comprehension of the invention and should not be construed to limit the scope thereof.

1.-19. (canceled)

20. An apparatus, comprising:
a proximal portion defining a channel extending therethrough and configured to convey a fluid, the proximal portion configured to be coupled to a source of ultrasonic energy; and
a blade disposed at a distal end of the proximal portion, the blade defining a through slot in fluid communication with the channel, the through slot extending longitudinally along a portion of the blade and configured to deliver the fluid to an area adjacent to the blade, the blade including a plurality of teeth disposed along at least a portion of an edge of the blade, the plurality of teeth configured to cut through biological tissue.

21. The apparatus of claim 20, wherein the blade includes a first lateral surface and a second lateral surface parallel to the first lateral surface, the through slot extending between the first lateral surface and the second lateral surface.

22. The apparatus of claim 20, wherein the channel is a first channel, the blade further includes a second channel co-linear with the first channel and configured to convey the fluid to the through slot.

23. The apparatus of claim 20, wherein the blade has a pair of longitudinal edges and a distal edge that is convexly arcuate and contiguous with the pair of longitudinal edges.

24. The apparatus of claim 23, wherein the plurality of teeth includes a first set of teeth and a second set of teeth different than the first set of teeth.

25. The apparatus of claim 24, wherein the first set of teeth is disposed on at least a portion of the distal edge of the blade, and the second set of teeth is disposed on at least a portion of the pair of longitudinal edges.

26. The apparatus of claim 25, wherein the first set of teeth have a first tooth length and the second set of teeth have a second tooth length, the first tooth length being smaller than the second tooth length.

27. The apparatus of claim 25, wherein the first set of teeth have a first pitch and the second set of teeth have a second pitch, the first pitch being smaller than the second pitch.

28. An apparatus, comprising:
a proximal portion defining a channel extending therethrough and configured to convey a fluid, the proximal portion configured to be coupled to a source of ultrasonic energy; and
a blade disposed at a distal end of the proximal portion, the blade including a first lateral surface and a second lateral surface parallel to the first lateral surface, the through slot extending between the first lateral surface to the second lateral surface, the through slot in fluid communication with the channel and configured to deliver the fluid to an area adjacent to the blade,
the blade further including a plurality of teeth disposed along at least a portion of an edge of the blade, the plurality of teeth configured to cut through biological tissue.

29. The apparatus of claim 28, wherein the blade has a pair of longitudinal edges and a distal edge that is convexly arcuate and contiguous with the pair of longitudinal edges.

30. The apparatus of claim 29, wherein the plurality of teeth includes a first set of teeth and a second set of teeth different than the first set of teeth.

31. The apparatus of claim 30, wherein the first set of teeth is disposed on at least a portion of the distal edge of the blade, and the second set of teeth is disposed on at least a portion of the pair of longitudinal edges.

32. The apparatus of claim 31, wherein the first set of teeth have a first tooth length and the second set of teeth have a second tooth length, the first tooth length being smaller than the second tooth length.

33. The apparatus of claim 31, wherein the first set of teeth have a first pitch and the second set of teeth have a second pitch, the first pitch being smaller than the second pitch.

34. The apparatus of claim 31, wherein a tooth length of the first set of teeth varies from a minimum an extreme at distal tip of the distal edge and increases gradually towards a respective longitudinal edge of the pair of longitudinal edges.

35. The apparatus of 31, wherein a tooth length of the first set of teeth is uniform among the first set of teeth and a tooth length of the second set of teeth is uniform among the second set of teeth.

36. An apparatus, comprising:
a proximal portion including a channel extending therethrough, the proximal portion configured to be coupled to a source of ultrasonic mechanical vibrations;
a blade disposed at a distal end of the proximal portion, the blade including a pair of longitudinal edges and a distal edge continuous with the pair of longitudinal edges, the blade defining a through slot extending along a portion thereof,
the blade further including a plurality of teeth including a first set of teeth disposed along at least a portion of the distal edge, and a second set of teeth disposed along at least a portion of the pair of longitudinal edges, the first set of teeth having a first length and a first pitch and the second set of teeth having a second length greater than the first length and a second pitch greater than the first pitch, the plurality of teeth configured to cut through biological tissue.

37. The apparatus of claim 36, wherein the channel is configured to convey a fluid, the through slot of the blade being in fluid communication with the channel and configured to deliver the fluid to an area adjacent to the blade.

38. The apparatus of claim 36, wherein the blade includes a first lateral surface and a second lateral surface parallel to the first lateral surface.

39. The apparatus of claim 38, the through slot extends between the first lateral surface and the second lateral surface.

40. The apparatus of claim 36, wherein the through slot extends longitudinally along a portion of the blade.

41. The apparatus of claim 36, wherein the plurality of teeth are a continuous array of teeth along the distal edge and the pair of longitudinal edges.

42. The apparatus of claim 36, wherein a tooth length of the first set of teeth varies from a minimum an extreme at distal tip of the distal edge and increases gradually towards a respective longitudinal edge of the pair of longitudinal edges.

43. The apparatus of 36, wherein a tooth length of the first set of teeth is uniform among the first set of teeth and a tooth length of the second set of teeth is uniform among the second set of teeth.
